(19) United States
(12) Patent Application Publication
(10) Pub. No.: US 2025/0145883 A1
(43) Pub. Date: May 8, 2025

(54) CORE-SHELL QUANTUM DOT, QUANTUM DOT LIGHT-EMITTING DEVICE, DISPLAY APPARATUS AND MANUFACTURING METHOD

(71) Applicants: Beijing BOE Technology Development Co., Ltd., Beijing (CN); BOE Technology Group Co., Ltd., Beijing (CN)

(72) Inventor: Wenhai MEI, Beijing (CN)

(21) Appl. No.: 18/578,675

(22) PCT Filed: Jul. 1, 2022
(86) PCT No.: PCT/CN2022/103427

§ 371 Date: Jan. 11, 2024

(30) Foreign Application Priority Data
Jul. 23, 2021 (CN) 202110835520.4

(51) Int. Cl. C09K 11/02; H10K 50/115

(52) U.S. Cl. CPC C09K 11/025; H10K 50/115; C09K 2211/18

(57) ABSTRACT

Disclosed are a core-shell quantum dot, a quantum dot light-emitting device, a display apparatus and a manufacturing method. The core-shell quantum dot includes: a core part; a shell layer arranged on at least part of a surface of the core part, and including metal ions; and a chelating ligand connected with the metal ions, where the chelating ligand and the metal ions form a closed ring structure connected with the shell layer.

### CROSS-REFERENCE TO RELATED APPLICATIONS

This application claims the priority of Chinese patent application No. 202110835520.4, filed to China National Intellectual Property Administration on Jul. 23, 2021 and entitled “Core-Shell Quantum Dot, Quantum Dot Light-Emitting Device, Display Apparatus and Manufacturing Method”, the entire contents of which are incorporated herein by reference.

### TECHNICAL FIELD

The present disclosure relates to the technical field of semiconductors, in particular to a core-shell quantum dot, a quantum dot light-emitting device, a display apparatus and a manufacturing method.

### BACKGROUND

A quantum dot light emitting diode display (QLED) is a new display technology developed based on an organic light-emitting display. The difference between the QLED and the organic light-emitting display is that a light-emitting layer in the QLED is a quantum dot layer, and a principle of the QLED is that electrons/holes are injected into the quantum dot layer through an electron/hole transport layer, and the electrons and holes are combined in the quantum dot layer to emit light. Compared with an organic light-emitting diode display device (OLED), the QLED has the advantages of a narrow emission peak, a high color saturation, a wide color gamut and the like.

On the other hand, with deep development of a quantum dot technology, the research of quantum dot display is increasingly deep, the quantum efficiency is continuously improved, which has basically reached the level of industrialization, and further adoption of new processes and technologies to achieve its industrialization has become a trend in the future.

### SUMMARY

Embodiments of the present disclosure provide a core-shell quantum dot, including:
a core part;
a shell layer, arranged on at least part of a surface of the core part, and including metal ions; and
a chelating ligand, connected with the metal ions,
where, the chelating ligand and the metal ions form a closed ring structure connected with the shell layer.

In some embodiments, a general formula of the chelating ligand includes:

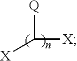

where n=2 or n=3; X represents a coordination group connected with the metal ions; and Q includes hydrogen or

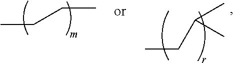

1≤m≤6, and 1≤r≤6.

In some embodiments, the chelating ligand includes a plurality of Q groups, and different Q groups include different groups.

In some embodiments, Q is a solubilizing group.

In some embodiments, the coordination group includes one or a combination of:
amino;
sulfhydryl;
carboxyl;
phosphoxy; or
hydroxy.

In some embodiments, the chelating ligand includes one or a combination of the following structural formulae:

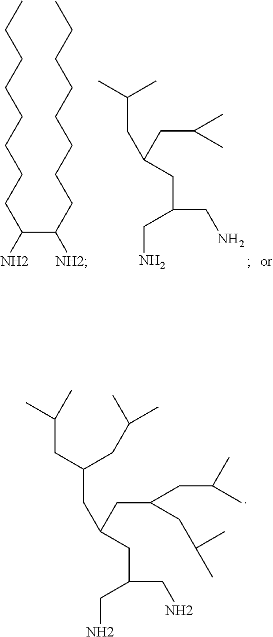

In some embodiments, two of the coordination groups coordinate with the same metal ion, and one of the core-shell quantum dots only coordinates with two of the coordination groups through the metal ion.

In some embodiments, the chelating ligand is of an axisymmetric structure.

In some embodiments, the metal ions include Group IIA metal ions, Group IIB metal ions or Group IA metal ions.

In some embodiments, the metal ions include one or a combination of beryllium ions, magnesium ions, barium ions, strontium ions, calcium ions, zinc ions, mercury ions, cadmium ions, gold ions, silver ions, copper ions, manganese ions, lead ions, tin ions, iron ions, or indium ions.

In some embodiments, one end of the chelating ligand facing away from the shell layer is further connected with at least one photoactive group.

In some embodiments, the photoactive group includes

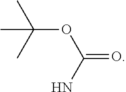

Embodiments of the present disclosure further provide a quantum dot light-emitting device, including a quantum dot film layer, wherein the quantum dot film layer includes the core-shell quantum dot provided by the embodiments of the present disclosure.

In some embodiments, one end of the chelating ligand facing away from the shell layer is connected with at least one —NH₂.

Embodiments of the present disclosure further provide a display apparatus, including the quantum dot light-emitting device provided by the embodiments of the present disclosure.

Embodiments of the present disclosure further provide a manufacturing method for a core-shell quantum dot, including:
adding quantum dots connected with a first ligand, and a metal ion-containing compound into a first solvent;
carrying out a first reaction to replace the first ligand with the metal ions to obtain quantum dots with a shell layer including the metal ions;
dissolving the quantum dots in a second solvent, and adding a chelating ligand into the second solvent; and
carrying out a second reaction to connect the chelating ligand with the metal ions to obtain quantum dots connected with the chelating ligand through the metal ions.

In some embodiments, the carrying out the first reaction to replace the first ligand with the metal ions to obtain the quantum dots with the shell layer including the metal ions, includes:
carrying out a reaction at a temperature ranging from 100° C. to 200° C. for 10 min to 30 min; and
precipitating a liquid after the reaction in a third solvent, performing centrifugation, and removing a supernatant to obtain a precipitate, and repeatedly performing the above operations for multiple times to obtain the quantum dots with the shell layer including the metal ions.

In some embodiments, the carrying out the second reaction to connect the chelating ligand with the metal ions to obtain the quantum dots connected with the chelating ligand through the metal ions, includes:
carrying out a reaction at room temperature for 3 h to 5 h; and
adding a fourth solvent, performing centrifugation, and removing a supernatant to obtain a precipitate, and repeatedly performing the above operations for multiple times to obtain the quantum dots connected with the chelating ligand through the metal ions.

### BRIEF DESCRIPTION OF FIGURES

FIG. 1 is a first structural schematic diagram of a core-shell quantum dot according to an embodiment of the present disclosure.

FIG. 2 is a second structural schematic diagram of the core-shell quantum dot according to the embodiment of the present disclosure.

FIG. 3 is a third structural schematic diagram of the core-shell quantum dot according to the embodiment of the present disclosure.

FIG. 4 is a fourth structural schematic diagram of the core-shell quantum dot according to the embodiment of the present disclosure.

FIG. 5 is a fifth structural schematic diagram of the core-shell quantum dot according to the embodiment of the present disclosure.

FIG. 6 is a sixth structural schematic diagram of the core-shell quantum dot according to the embodiment of the present disclosure.

FIG. 7 is a seventh structural schematic diagram of the core-shell quantum dot according to the embodiment of the present disclosure.

FIG. 8 is a comparative schematic diagram of conventional quantum dots and quantum dots provided by the embodiment of the present disclosure in a patterning process.

FIG. 9 is a comparative schematic diagram of conventional quantum dots and quantum dots provided by the embodiment of the present disclosure during operation of a device.

FIG. 10 is a schematic flow diagram of manufacture of a core-shell quantum dot according to an embodiment of the present disclosure.

### DETAILED DESCRIPTION

In order to make the objectives, technical solutions, and advantages of embodiments of the present disclosure clearer, the technical solutions of embodiments of the present disclosure will be described clearly and completely below with reference to the accompanying drawings of embodiments of the present disclosure. Obviously, the described embodiments are a part of embodiments of the present disclosure, but not all of embodiments. Based on the described embodiments of the present disclosure, all other embodiments obtained by those of ordinary skill in the art without inventive efforts fall within the scope of protection of the present disclosure.

Unless otherwise defined, the technical or scientific terms used in the present disclosure shall have the ordinary meaning as understood by those of ordinary skill in the art to which the present disclosure belongs. The terms “first,” “second,” and the like as used in the present disclosure do not denote any order, quantity, or importance, but are merely used to distinguish different components. “Include” or “comprise” and other similar words mean that an element or item preceding the word covers elements or items listed behind the word and their equivalents without excluding other elements or items. “Connection” or “connected” and the like are not limited to physical or mechanical connection, but can include electrical connection, whether direct or indirect. “Upper”, “lower”, “left”, “right” and the like are only used for representing a relative position relation, and when an absolute position of the described object is changed, the relative position relation can also be correspondingly changed.

In order to keep the following description of embodiments of the present disclosure clear and concise, the present disclosure omits detailed descriptions of known functions and known components.

Patterning quantum dots to manufacture high-resolution QLED or QD-LCD has become an important issue, but residues can be easily formed after a development process in the current process for directly patterning quantum dots, easily causing the color mixing problem in full-color quantum dot display. Meanwhile, during the operation of the QLED device, ligands are prone to detachment to cause defects on the surfaces of the quantum dots, resulting in roll-off of the device efficiency, and the like.

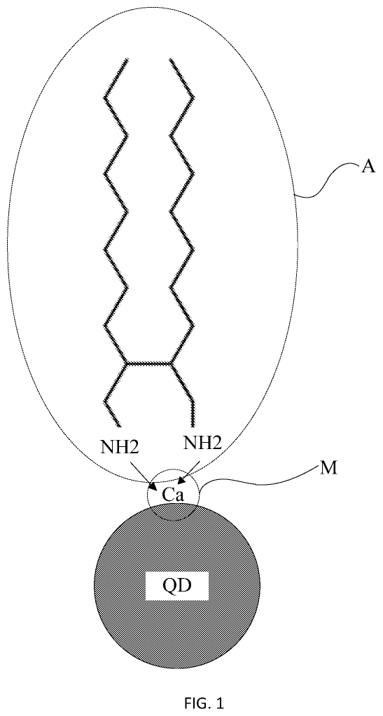

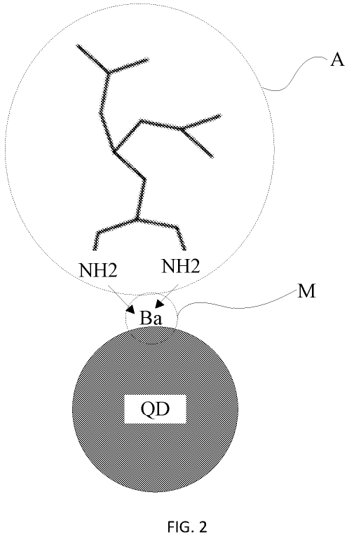

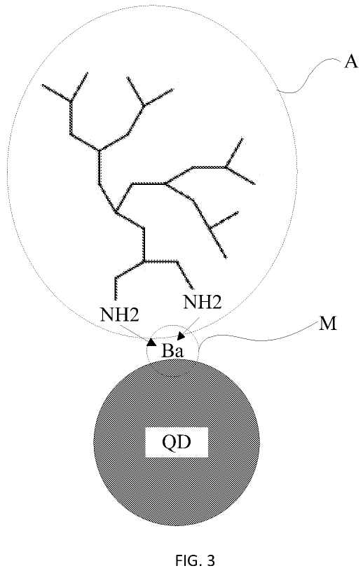

As shown in FIG. 1, FIG. 2, and FIG. 3, an embodiment of the present disclosure provides a core-shell quantum dot, including:
a core part;
a shell layer, arranged on at least part of a surface of the core part, and including metal ions M; and
a chelating ligand A connected with the metal ions M, where the chelating ligand A and the metal ions M form a closed ring structure connected with the shell layer.

According to the core-shell quantum dot provided by the embodiment of the present disclosure, metal ions are introduced into the shell layer of the quantum dot, and then a chelating ligand and the metal ions introduced form a stable closed ring structure (a five- or six-membered ring structure), so that the adsorption capacity of the ligand on the surface of the quantum dot can be increased. When a patterned quantum dot film layer is manufactured by using the core-shell quantum dot provided by the embodiment of the present disclosure, the problem that the conventional ligand is easily detached in a patterned development process, resulting in the residue of a quantum dot film layer in this removed region, causing color mixing in full-color quantum dot display can be avoided. In a quantum dot device manufactured by using the core-shell quantum dot provided by the embodiment of the present disclosure, ligands are not easily detached on the surface of the quantum dot, and the problem that the conventional ligand is easily detached, resulting in defects on the surfaces of the quantum dots and resulting in reduced efficiency of the device can also be avoided.

In some embodiments, a general formula of the chelating ligand A may include:

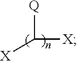

where n=2 or n=3; X represents a coordination group connected with the metal ions; Q may be a solubilizing group, and in particular may include hydrogen, or

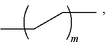

1≤m≤6, or

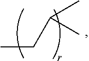

1≤r≤6. n=2 or n=3, so that the chelating ligand A and metal ions may form a stable five- or six-membered ring structure, which is not easily broken and decomposed, and a ligand with strong adsorption capacity to the shell layer is formed. Moreover, the more the number of Q as a dissolution unit, the better the solubility, but in consideration of carrier transport properties, too much of m and r tends to cause too strong insulating properties. Therefore, in embodiments of the present disclosure, 1≤m≤6 and 1≤r≤6, so that the chelating ligand A can have better carrier transport properties while having better solubility.

In some embodiments, the chelating ligand A includes a plurality of Q groups, and different Q groups include different groups.

In some embodiments, the general formula of the chelating ligand A may include one of:

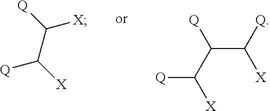

In some embodiments, the coordination group X includes one or a combination of: amino, sulfhydryl, carboxyl, phosphoxy, or hydroxy. In this way, better properties of connection with the metal ions are achieved.

In some embodiments, the chelating ligand A includes one or a combination of the following structural formulae:

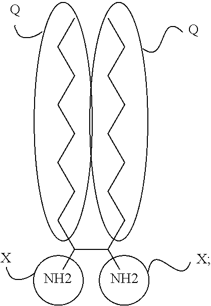

where two Q are included, and the two Q are linear alkyl chains;

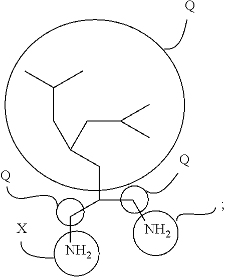

where three Q are included, one Q is a branched alkyl chain and two Q are H; or

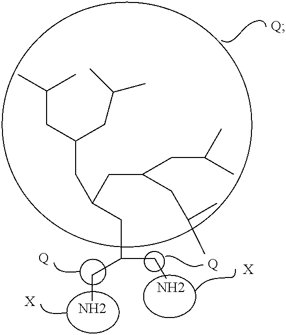

where three Q are included, one Q is a branched alkyl chain and two Q are H.

In some embodiments, two of the coordination groups X coordinate with the same metal ion, and one of the core-shell quantum dot only coordinates with two of the coordination groups X through the metal ion M. In this way, while reducing the probability of ligand detachment, it is avoided that if the chelating ligand A has more than two coordination groups, it is easy for the chelating ligand A to coordinate with a plurality of quantum dots simultaneously, resulting in quantum dot coagulation.

In some embodiments, the chelating ligand A is of an axisymmetric structure.

In some embodiments, the metal ions M may include Group IIA metal ions, Group IIB metal ions or Group IA metal ions. In some embodiments, the metal ions M may include one or a combination of: beryllium ions, magnesium ions, barium ions, strontium ions, calcium ions, zinc ions, mercury ions, cadmium ions, gold ions, silver ions, copper ions, manganese ions, lead ions, tin ions, iron ions, or indium ions.

In some embodiments, the core-shell quantum dot includes one or a combination of:

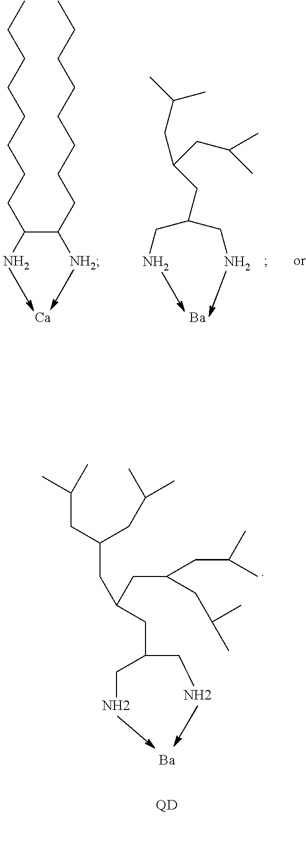

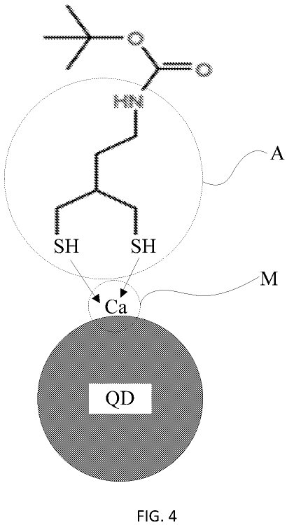

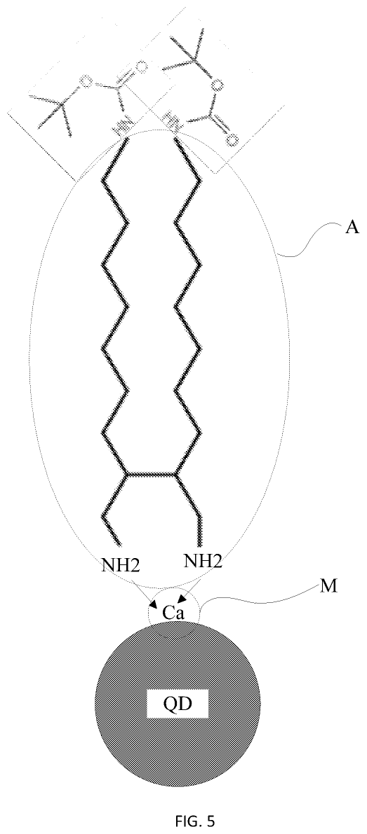

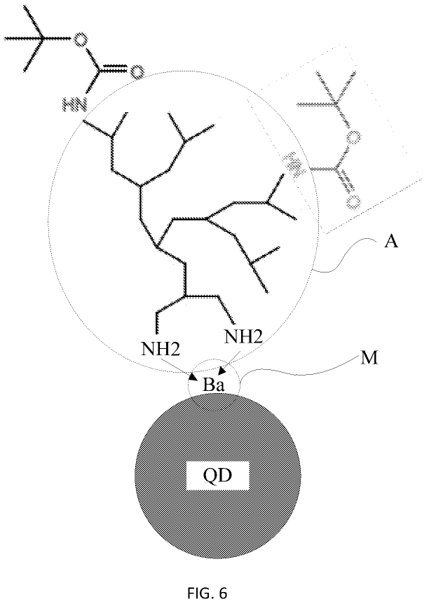

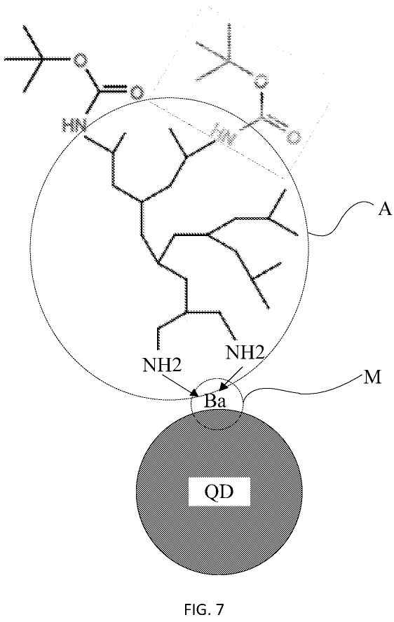

In some embodiments, one end of the chelating ligand A facing away from the shell layer is further connected with at least one photoactive group. Referring to FIGS. 4, 5, 6, and 7, the photoactive group includes:

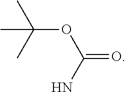

In some embodiments, the photoactive groups may also be other types of groups. Thus, when a patterned quantum dot film layer is formed by a photolithography process by using the core-shell quantum dot provided by the embodiment of the present disclosure, a photo acid generator capable of generating hydrogen ions upon irradiation with ultraviolet light is mixed in a quantum dot thin film, then, in an area irradiated by ultraviolet light,

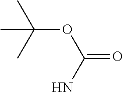

is converted to —NH2, while in an area not irradiated by ultraviolet light,

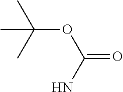

remains, and then a quantum dot thin film in an area including

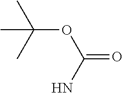

can be removed by the selection of a solvent, while a quantum dot thin film in an area including —NH2 remains, the patterning of the quantum dot film layer can be achieved, and a pattern formed can be made more precise.

It can be understood that when one end of the chelating ligand A facing away from the shell layer is also connected with at least one

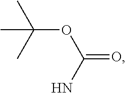

a structure connected with one end of the chelating ligand A facing away from the shell layer in the finally retained quantum dot film layer is —NH2 when the patterned quantum dot film layer is formed by the photolithography process. Of course, in some embodiments, if the patterned quantum dot film layer is not formed by photolithography, for example, the patterned quantum dot film layer is formed by inkjet printing, the chelating ligand A, and

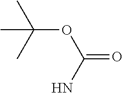

connected with one end of the chelating ligand A facing away from the shell layer may also be included in the finally formed patterned quantum dot film layer. Of course, if

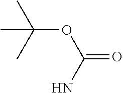

is not connected with one end of the chelating ligand A facing away from the shell layer, the patterned quantum dot film layer is formed directly by an inkjet printing process, and the finally formed patterned quantum dot film layer may include the chelating ligand A, not including

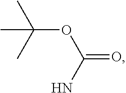

and also not including —NH2.

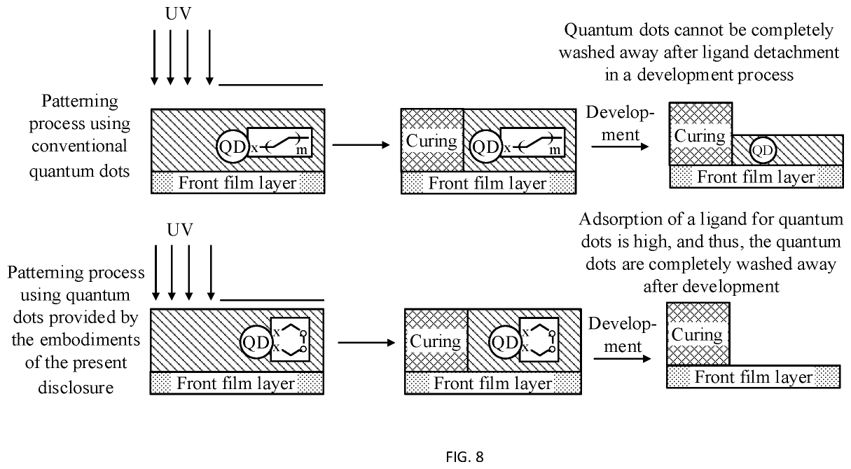

In some embodiments, as shown in FIG. 8, a schematic diagram of conventional quantum dots and quantum dots provided by the embodiment of the present disclosure in a patterning process is shown. In a conventional quantum dot patterning process, in an area irradiated by ultraviolet light, ligands are separated from quantum dots; in an area not irradiated by ultraviolet light, the ligands should be connected with the quantum dots. But due to the weak adsorption force of conventional ligands to quantum dots, in the patterning process, the ligands are also separated from the quantum dots, and then, in the subsequent development cleaning process, in the area irradiated by ultraviolet light, the quantum dots remain, while in the area not irradiated by ultraviolet light, the ligands are also separated from the quantum dots, the quantum dots also remain, causing the residue of the quantum dots in this removed region, and causing the problem of color mixing in full-color quantum dot display. When the quantum dot film layer is patterned by using the core-shell quantum dot provided by the embodiment of the present disclosure, in the area irradiated by ultraviolet light, the metal ions in the shell layer are separated from the chelating ligand

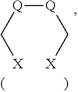

in the area not irradiated by ultraviolet light, the chelating ligand is still connected with the shell layer of the core-shell quantum dot through the metal ions, in the process of development cleaning, by selecting a solvent capable of dissolving the chelating ligand, the quantum dots connected with the chelating ligand in the area not irradiated by ultraviolet light can be washed away together, while the quantum dots in the area irradiated by ultraviolet light remain, and thus, a precise patterned quantum dot film layer is obtained. Or, in the core-shell quantum dot according to the embodiment of the present disclosure, when one end of the chelating ligand facing away from the shell layer is further connected with

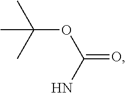

in the area irradiated by ultraviolet light,

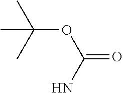

interacts with hydrogen ions, and is partially broken under the action of a photo acid generator (the photo acid generator can generate hydrogen ions upon irradiation with ultraviolet light), and a moiety connected with the chelating ligand is converted to —NH2; while in the area not irradiated by ultraviolet light,

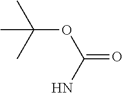

remains, and then the quantum dot thin film including the area

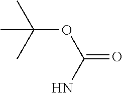

can be removed by the selection of a solvent, while the quantum dot thin film including the area —NH2 remains, the patterning of the quantum dot film layer can be achieved, and the pattern formed can be made more precise.

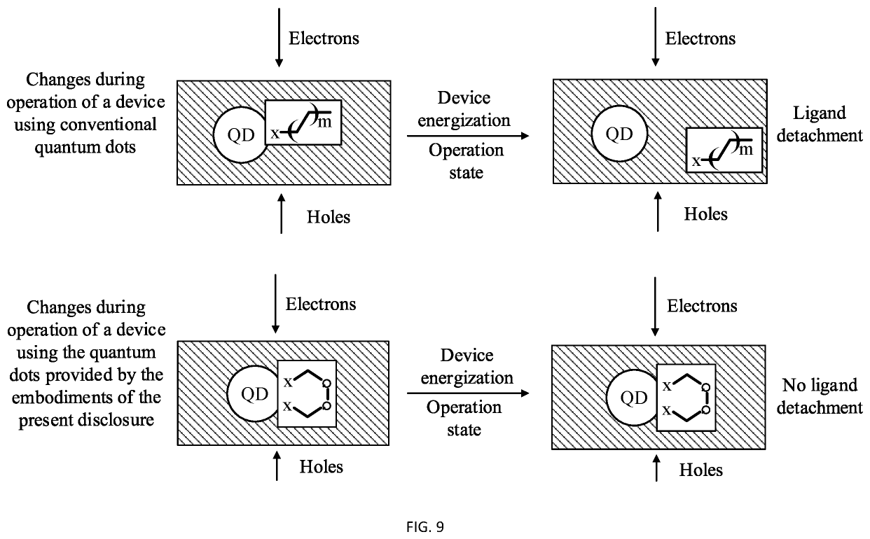

In some embodiments, as shown in FIG. 9, a schematic diagram of conventional quantum dots and quantum dots provided by the embodiment of the present disclosure during operation of a device is shown. The conventional ligands are easily detached, resulting in defects on the surfaces of the quantum dots, resulting in reduced efficiency of the device. However, in a quantum dot device manufactured by using the core-shell quantum dot provided by the embodiment of the present disclosure, ligands are not easily detached on the surface of the quantum dot, and the device efficiency is stable.

It should be noted that in a lower graph of FIG. 8 and in a lower graph of FIG. 9, metal ions are connected between the quantum dot and the chelating ligand.

Based on the same inventive concept, an embodiment of the present disclosure further provides a quantum dot light-emitting device, including a quantum dot film layer, where the quantum dot film layer includes the core-shell quantum dot provided by the embodiments of the present disclosure.

In some embodiments, one end of the chelating ligand A facing away from the shell layer is connected with at least one —NH2.

Based on the same inventive concept, an embodiment of the present disclosure further provides a display apparatus, including the quantum dot light-emitting device provided by the embodiments of the present disclosure.

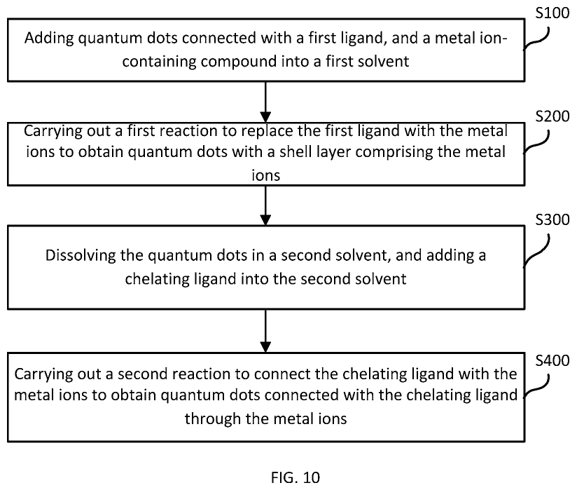

Based on the same inventive concept, an embodiment of the present disclosure further provides a manufacturing method for a core-shell quantum dot, referring to FIG. 10, including the following steps.

Step S100, adding quantum dots connected with a first ligand, and a metal ion-containing compound into a first solvent. In some embodiments, the first solvent may be octadecene, the quantum dots may be CdSe/ZnS quantum dots, the first ligand may be oleic acid, and the metal ion-containing compound may be calcium chloride or barium chloride.

Step S200, carrying out a first reaction to replace the first ligand with the metal ions to obtain quantum dots with a shell layer including the metal ions. In some embodiments, this step may include:
carrying out a reaction at a temperature ranging from 100° C. to 200° C. for 10 min to 30 min; and
precipitating a liquid after the reaction in a third solvent, performing centrifugation, and removing a supernatant to obtain a precipitate, and repeatedly performing the above operations for multiple times to obtain the quantum dots with the shell layer including the metal ions; in particular, the third solvent may be methanol.

Step S300, dissolving the quantum dots in a second solvent, and adding a chelating ligand into the second solvent. In some embodiments, the second solvent may be toluene.

Step S400, carrying out a second reaction to connect the chelating ligand with the metal ions to obtain quantum dots connected with the chelating ligand through the metal ions. In some embodiments, this step may include:
carrying out a reaction at room temperature for 3 h to 5 h; and
adding a fourth solvent, performing centrifugation, and removing a supernatant to obtain a precipitate, and repeatedly performing the above operations for multiple times to obtain the quantum dots connected with the chelating ligand through the metal ions. In some embodiments, the fourth solvent may be octane.

Manufacturing processes for different quantum dot light-emitting devices are provided as below.

A manufacturing process of an upright QLED device with quantum dots containing a chelating ligand is provided as below.

1. Quantum dots containing calcium ions are prepared by ion exchange:

30 mg of CdSe/ZnS quantum dots with an oleic acid ligand on the surfaces thereof, and 5 mg of calcium chloride are dissolved in 5 ml of octadecene, heating is performed to 150° C., a reaction is carried out for 20 minutes, completing ligand exchange, the resulting reaction solution is precipitated in 50 ml of methanol, centrifugation is performed, a supernatant is removed to obtain quantum dots of which a shell layer contains calcium ions, precipitation and centrifugation are repeated, and the quantum dots are washed for three times, and dissolved in toluene to form a 15 mg/ml solution for standby application.

2. Quantum dots containing a chelating cyclic ligand are obtained by ligand exchange:

1 mL of the quantum dot toluene solution formed in the step 1 is mixed with 100 mg of a chelating ligand A for ligand exchange, the quantum dots are precipitated in methanol after a reaction for 4 hours, centrifugation is performed, a supernatant is removed to obtain quantum dots, precipitation and centrifugation are repeated, and the quantum dots are washed for three times, and dissolved in octane to form a 15 mg/ml solution for standby application.

3. A hole injection layer is manufactured on a base substrate including an anode layer (a specific material may be indium tin oxide), specifically, PEDOT:PSS may be spin-coated in air (4000 rpm, 30 s), and annealing is performed at 230° C. for 20 min; a hole transport layer is manufactured, specifically, a TFB solution may be spin-coated (3000 rpm, 30 s) in a glove box, and annealing is performed at 150° C. for 15 min; a quantum dot layer is manufactured, specifically, the quantum dot octane solution prepared in the step 2 may be spin-coated (2500 rpm, 30 s), and annealing is performed at 120° C. for 20 min; an electron transport layer is manufactured, specifically, a zinc oxide nanoparticle solution may be spin-coated (2000 rpm, 30 s), and annealing is performed at 120° C. for 20 min; and a cathode layer (e.g., a material of the cathode layer is aluminum) of 120 nm is evaporated, and encapsulation is performed, completing device manufacture.

A manufacturing process of an inverted QLED device with quantum dots containing a chelating ligand is provided as below.

1. Quantum dots containing barium ions are prepared by ion exchange:

30 mg of CdSe/ZnS quantum dots, a ligand being oleic acid, and 5 mg of barium chloride are dissolved in 5 ml of octadecene, heating is performed to 150° C., a reaction is carried out for 20 minutes, completing ligand exchange, the resulting reaction solution is precipitated in 50 ml of methanol, centrifugation is performed, a supernatant is removed to obtain quantum dots of which a shell layer contains barium ions, precipitation and centrifugation are repeated, and the quantum dots are washed for three times, and dissolved in toluene to form a 15 mg/ml solution for standby application.

2. Quantum dots containing a chelating cyclic ligand are obtained by ligand exchange:

1 mL of the quantum dot toluene solution formed in the step 1 is mixed with 100 mg of a chelating ligand B for ligand exchange, the quantum dots are precipitated in methanol after a reaction for 4 hours, centrifugation is performed, a supernatant is removed to obtain quantum dots, precipitation and centrifugation are repeated, and the quantum dots are washed for three times, and dissolved in octane to form a 15 mg/ml solution for standby application.

3. An electron transport layer is manufactured on a base substrate including a cathode layer (a specific material may be indium tin oxide), specifically, a zinc oxide nanoparticle solution may be spin-coated (2000 rpm, 30 s) in a glove box, and annealing is performed at 120° C. for 20 minutes; a quantum dot layer is manufactured, specifically, the quantum dot octane solution prepared in the step 2 is spin-coated (2500 rpm, 30 s), and annealing is performed at 120° C. for 20 minutes; a hole transport layer material and a hole injection layer material which are 50 nm in total are evaporated; an anode layer (specifically, a material may be silver) of 120 nm is evaporated, and encapsulation is performed, completing device manufacture.

A manufacturing process of a patterned QLED device with quantum dots containing a chelating ligand is provided as below.

1. Quantum dots containing barium ions are prepared by ion exchange:

30 mg of CdSe/ZnS red quantum dots, a ligand being oleic acid, and 5 mg of barium chloride are dissolved in 5 ml of octadecene, heating is performed to 150° C., a reaction is carried out for 20 minutes, completing ligand exchange, the resulting reaction solution is precipitated in 50 ml of methanol, centrifugation is performed, a supernatant is removed to obtain red quantum dots of which a shell layer contains barium ions, precipitation and centrifugation are repeated, and the quantum dots are washed for three times, and dissolved in toluene to form a 15 mg/ml solution for standby application. Green and blue quantum dots are prepared according to the same process.

2. Quantum dots containing a chelating cyclic ligand are obtained by ligand exchange:

1 ml of the quantum dot toluene solution formed in the step 1 is mixed with 100 mg of a chelating ligand C for ligand exchange, the quantum dots are precipitated in methanol after a reaction for 4 hours, centrifugation is performed, a supernatant is removed to obtain quantum dots, precipitation and centrifugation are repeated, and the quantum dots are washed for three times, and dissolved in toluene, and a photo acid generator (PAG) with a mass fraction of 5% is added to form a 15 mg/ml solution for standby application. Red, green and blue quantum dots can all be prepared according to this process.

3. An electron transport layer is manufactured on a base substrate including a cathode layer (a specific material may be indium tin oxide), specifically, a zinc oxide nanoparticle solution may be spin-coated (2000 rpm, 30 s) in a glove box, and annealing is performed at 120° C. for 20 minutes; the toluene solution of red quantum dots prepared in the step 2 (containing 2,4-bis(trichloromethyl)-6-p-methoxystyryl-S-triazine with a mass fraction of 5% as a photo acid generator) is spin-coated (2500 rpm, 30 s), and a quantum dot film layer is subjected to patterned exposure by using a mask plate, wherein an exposure wavelength is 365 nm, and the exposure amount is 100 mj; development is performed by using toluene for 120 s after exposure completion, and annealing is performed at 120° C. for 20 minutes to obtain a patterned red quantum dot film layer; patterned green and blue quantum dot film layers are also prepared according to this process; a hole transport layer material and a hole injection layer material which are 50 nm in total are evaporated; and a silver electrode of 120 nm is evaporated, and encapsulation is performed, completing device manufacture.

The beneficial effects of the embodiments of the present disclosure are as follows: according to the core-shell quantum dot provided by the embodiment of the present disclosure, the metal ions are introduced into the shell layer of the quantum dot, and then the chelating ligand and the metal ions introduced form a stable closed ring structure (a five- or six-membered ring structure), so that the adsorption capacity of the ligand on the surface of the quantum dot can be increased, when a patterned quantum dot film layer is manufactured by using the core-shell quantum dot provided by the embodiment of the present disclosure, the problem that the conventional ligand is easily detached in a patterned development process, resulting in the residue of a quantum dot film layer in this removed region, causing color mixing in full-color quantum dot display can be avoided; and in a quantum dot device manufactured by using the core-shell quantum dot provided by the embodiment of the present disclosure, ligands are not easily detached on the surface of the quantum dot, and the problem that the conventional ligand is easily detached, resulting in defects on the surfaces of the quantum dots and resulting in reduced efficiency of the device can also be avoided.

Obviously, those skilled in the art can make various modifications and variations to the present disclosure without departing from the spirit and scope of the present disclosure. Thus, if these modifications and variations of the present disclosure fall within the scope of the claims of the present disclosure and their equivalents, the present disclosure is also intended to include these modifications and variations.

1. A core-shell quantum dot, comprising:
a core part;
a shell layer, arranged on at least part of a surface of the core part, and comprising metal ions; and
a chelating ligand, connected with the metal ions;
wherein the chelating ligand and the metal ions form a closed ring structure connected with the shell layer.

2. The core-shell quantum dot according to claim 1, wherein a general formula of the chelating ligand comprises: 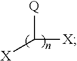
wherein n=2 or n=3; X represents a coordination group connected with the metal ions; and Q comprises hydrogen, or 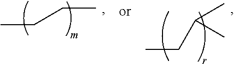
1≤m≤6, and 1≤r≤6.

3. The core-shell quantum dot according to claim 2, wherein the chelating ligand comprises a plurality of Q groups, and different Q groups comprise different groups.

4. The core-shell quantum dot according to claim 2, wherein Q is a solubilizing group.

5. The core-shell quantum dot according to claim 2, wherein the coordinating group comprises one or a combination of:
amino;
sulfhydryl;
carboxyl;
phosphoxy; or
hydroxy.

6. The core-shell quantum dot according to claim 2, wherein the chelating ligand comprises one or a combination of following structural formulae:

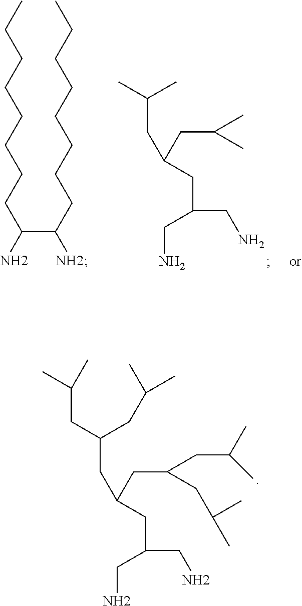 [SMILES: CCCCCCCCC(N)C(N)CCCCCCCC ; CC(C)CC(CC(C)C)CC(CN)CN ; CC(C)CC(CC(C)C)CC(CC(CN)CN)CC(CC(C)C)CC(C)C]

7. The core-shell quantum dot according to claim 2, wherein two of the coordination groups coordinate with a same metal ion, and one of the core-shell quantum dots only coordinates with two of the coordination groups through the metal ion.

8. The core-shell quantum dot according to claim 2, wherein the chelating ligand is of an axisymmetric structure.

9. The core-shell quantum dot according to claim 1, wherein the metal ions comprise Group IIA metal ions, Group IIB metal ions, or Group IA metal ions.

10. The core-shell quantum dot according to claim 9, wherein the metal ions comprise one or a combination of: beryllium ions, magnesium ions, barium ions, strontium ions, calcium ions, zinc ions, mercury ions, cadmium ions, gold ions, silver ions, copper ions, manganese ions, lead ions, tin ions, iron ions, or indium ions.

11. The core-shell quantum dot according to claim 1, wherein one end of the chelating ligand facing away from the shell layer is further connected with at least one photoactive group.

12. The core-shell quantum dot according to claim 11, wherein the photoactive group comprises

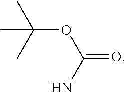

13. A quantum dot light-emitting device, comprising a quantum dot film layer, wherein the quantum dot film layer comprises the core-shell quantum dot according to claim 1.

14. The quantum dot light-emitting device according to claim 13, wherein one end of the chelating ligand facing away from the shell layer is connected with at least one —NH2.

15. A display apparatus, comprising the quantum dot light-emitting device according to claim 13.

16. A manufacturing method for a core-shell quantum dot, comprising:
adding quantum dots connected with a first ligand, and a metal ion-containing compound into a first solvent;
carrying out a first reaction to replace the first ligand with the metal ions to obtain quantum dots with a shell layer comprising the metal ions;
dissolving the quantum dots in a second solvent, and adding a chelating ligand into the second solvent; and
carrying out a second reaction to connect the chelating ligand with the metal ions to obtain quantum dots connected with the chelating ligand through the metal ions.

17. The manufacturing method according to claim 16, wherein the carrying out the first reaction to replace the first ligand with the metal ions to obtain the quantum dots with the shell layer comprising the metal ions, comprises:
carrying out a reaction at a temperature ranging from 100° C. to 200° C. for 10 min to 30 min; and
precipitating a liquid after the reaction in a third solvent, performing centrifugation, and removing a supernatant to obtain a precipitate, and repeatedly performing the above operations for multiple times to obtain the quantum dots with the shell layer comprising the metal ions.

18. The manufacturing method according to claim 16, wherein the carrying out the second reaction to connect the chelating ligand with the metal ions to obtain the quantum dots connected with the chelating ligand through the metal ions, comprises:
carrying out a reaction at room temperature for 3 h to 5 h; and
adding a fourth solvent, performing centrifugation, and removing a supernatant to obtain a precipitate, and repeatedly performing the above operations for multiple times to obtain the quantum dots connected with the chelating ligand through the metal ions.

19. The quantum dot light-emitting device according to claim 13, wherein a general formula of the chelating ligand comprises: 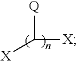
wherein n=2 or n=3; X represents a coordination group connected with the metal ions; and Q comprises hydrogen, or 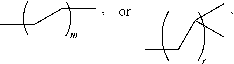
1≤m≤6, and 1≤r≤6.

20. The quantum dot light-emitting device according to claim 19, wherein the chelating ligand comprises a plurality of Q groups, and different Q groups comprise different groups.
